(19) United States
(12) Patent Application Publication
(10) Pub. No.: US 2025/0302845 A1
(43) Pub. Date: Oct. 2, 2025

(54) SELECTIVE DOPAMINE INCREASE

(71) Applicant: SOBRERA PHARMA AB, Göteborg (SE)

(72) Inventors: Bo SÖDERPALM, Bollebygd (SE); Klara DANIELSSON, Göteborg (SE); Rosita STOMBERG, Billdal (SE); Louise ADERMARK, Västra Frölunda (SE); Mia ERICSON, Göteborg (SE)

(21) Appl. No.: 18/863,395

(22) PCT Filed: May 8, 2023
(86) PCT No.: PCT/SE2023/050447

§ 371 Date: Nov. 6, 2024

Related U.S. Application Data
(60) Provisional application No. 63/340,567, filed on May 11, 2022.

(51) Int. Cl. A61K 31/55; A61K 31/166; A61K 31/198

(52) U.S. Cl. CPC A61K 31/55; A61K 31/166; A61K 31/198

(57) ABSTRACT

The invention relates to a combination comprising an effective amount of varenicline, or a salt thereof, and an effective amount of a glycine transport (GlyT) inhibitor. This combination achieves a selective dopamine increase in ventral striatum (nucleus accumbens) whilst not significantly altering dorsal striatum (associative striatum) dopamine. Such a selective dopamine elevation is of benefit for subjects suffering from schizophrenia. In particular, the combination of varenicline, or the salt thereof, and the GlyT transporter is useful for treatment of the negative symptoms in schizophrenia, without the risk of exacerbating the positive symptoms.

### TECHNICAL FIELD

The present invention relates to a combination of varenicline and a glycine transporter (GlyT) inhibitor, and the use thereof to achieve a selective dopamine (DA) increase in ventral striatum.

### BACKGROUND

Schizophrenia is a life-long and debilitating psychiatric disorder that affects roughly 1% of the global population. The condition is characterized by three main categories of symptoms, positive symptoms (e.g., hallucinations and delusions), negative symptoms (e.g., blunted affect, anhedonia and avolition), and cognitive symptoms (e.g., dysfunctional memory, lack of attention and executive dysfunction).

Most available pharmacological treatments for schizophrenia focus on the positive symptomatology and comprise first and second-generation anti-psychotics. These agents invoke dopamine (DA) D2-receptor antagonism, thereby blocking aberrant dopaminergic transmission in the brain. This connection between reduced dopaminergic signaling and reduced positive symptomatology led to the formation of the so called “dopamine hypothesis”, which, in its original form stipulates that a hyperdopaminergic state results in schizophrenia. This hypothesis has since been modified, mainly due to the fact that not only are the negative symptoms largely unaffected by traditional D2-receptor antagonism; they can actually be worsened by the subsequent reduction in dopaminergic transmission. The modified dopamine hypothesis postulates that there is in fact a discrepancy in DA signaling that contributes to the symptomatology of schizophrenia, namely that while a hyperdopaminergic state in the dorsal striatum (DS), more specifically in the associative striatum, produces the positive symptoms, the negative and cognitive symptoms may in fact result from a hypodopaminergic state in other regions of the brain, primarily the prefrontal cortex and the ventral striatum, which is supported by imaging studies suggesting that negative symptomatology is related to the ventral, rather than the dorsal striatum. This modified hypothesis, and the fact that many individuals with schizophrenia report that the negative and cognitive symptoms have a substantial negative impact on their quality of life, highlights the need for new treatments specifically tailored towards the treatment of negative symptoms.

There is, thus, a need for an effective treatment of schizophrenia and in particular a treatment of negative symptoms of schizophrenia that does not exacerbate positive symptoms of schizophrenia.

### SUMMARY

It is a general objective to provide a selective dopamine elevation in a subject.

It is a particular objective to provide an effective treatment of schizophrenia.

It is another particular objective to provide a treatment of negative symptoms of schizophrenia that does not exacerbate positive symptoms of schizophrenia

These and other objectives are met by embodiments of the invention.

An aspect of the invention relates to a combination comprising an effective amount of varenicline, or a salt thereof, and an effective amount of a glycine transport (GlyT) inhibitor.

Other aspects of the invention relate to a combination according to above for use as a medicament, for use in treatment of schizophrenia, or for use in treatment of negative symptoms of schizophrenia in a subject suffering from schizophrenia.

The combination of varenicline, or the salt thereof, and the GlyT inhibitor achieves a selective dopamine increase in ventral striatum (nucleus accumbens) whilst not significantly altering dorsal striatum dopamine. Such a selective dopamine elevation is of benefit for subjects suffering from schizophrenia. In particular, the combination of varenicline, or the salt thereof, and the GlyT transporter is useful for treatment of the negative symptoms in schizophrenia, without the risk of exacerbating the positive symptoms.

### BRIEF DESCRIPTION OF THE DRAWINGS

The embodiments, together with further objectives and advantages thereof, may best be understood by making reference to the following description taken together with the accompanying drawings, in which:

FIG. 1: Photographic illustration of the custom-made double probe used in the second set of experiments. Schematic illustration of the probe placement, as determined by macroscopic observation. Both single and double probe placements are included in the figure.

FIG. 2: Combination of ethanol and nicotine produces an enhanced effect in the nucleus accumbens (nAc) but not in the dorsomedial striatum (DMS). A) Nicotine (Nic, 0.36 mg/kg) alone and in combination with ethanol (EtOH, 2.5 g/kg) both produce a significant and substantial increase in accumbal DA, whereas the more transient effect seen from ethanol alone was not statistically significant in the area under the curve (AUC) analysis. The combination of nicotine and ethanol potentiated the effect of either treatment alone. B) Both ethanol and nicotine alone elevated DA significantly, as compared to vehicle treated controls (Veh). However, the combination of the treatments did not significantly differ from the vehicle group, and the addition of ethanol instead seemed to attenuate the nicotine-induced DA elevation in the DMS. C) Ethanol produced a transient, albeit non-significant, elevation in DA, that did not differ between the nAc and the DMS. However, a statistically significant interaction factor suggests that ethanol affects dopaminergic transmission in the two areas differently over time. D) Similarly to ethanol, nicotine failed to produce quantitatively different DA elevations in the nAc and DMS, but did affect the two regions differently over time. E) The combination of ethanol and nicotine produced a significantly and substantially more robust elevation of accumbal DA, as compared to the effects seen in the DMS. All values are presented as means±SEM. n=number of rats. *p<0.05, **p<0.01, ***p<0.001.

FIG. 3: Enhanced effect of the combination of Org 24598 and varenicline in the nAc but not in DMS. A) The combination of Org 24598 (9 mg/kg) and varenicline (Var, 1.5 mg/kg) significantly elevated nAc DA, as compared to vehicle treated controls (Veh). B) In the DMS, varenicline produced a significant increase in extracellular DA. However, this effect was tempered by the addition of Org 24598, as the combination of the two did not manage to alter DA levels. C) Org 24598 did not have a significantly differential effect on nAc and DMS DA. D) While varenicline produced a significant effect on both nAc and DMS DA over time, this effect did not differ between the two brain regions. E) The combination of Org 24598 and varenicline gave rise to significantly different effects in the nAc and the DMS, with a substantially more robust and prolonged DA elevation in the nAc. All values are presented as means±SEM. n number of rats. ***p<0.001.

FIG. 4: The GlyT2 inhibitor Org 25543 displayed a dose-dependent increase of dopamine in the nucleus accumbens following systemic administration (4 or 8 mg/kg i.p.). The drug was administered at time-point 0 as indicated by the arrow.

FIG. 5: The GlyT2 inhibitor Org 25543 was administered systemically (12 mg/kg i.p.) and dopamine levels were monitored simultaneously in the nucleus accumbens and the dorsomedial striatum in the same Wistar rat.

### DETAILED DESCRIPTION

Schizophrenia is a mental disorder characterized by continuous or relapsing episodes of psychosis. Symptoms are described in terms of positive symptoms, negative symptoms, and cognitive symptoms (National Institute of Mental Health, Schizophrenia, www.nimh.nih.gov/health/topics/schizophrenia).

Positive symptoms, also referred to as psychotic symptoms in the art, include changes in the way a person thinks, acts, and experiences the world. People with positive symptoms may lose a shared sense of reality with others and experience the world in a distorted way. Positive symptoms include:
Hallucinations: When a person sees, hears, smells, tastes, or feels things that are not actually there. Hearing voices is common for people with schizophrenia.
Delusions: When a person has strong beliefs that are not true and may seem irrational to others. For example, individuals experiencing delusions may believe that people on the radio and television are sending special messages that require a certain response, or they may believe that they are in danger or that others are trying to hurt them.
Thought disorder: When a person has ways of thinking that are unusual or illogical. People with thought disorder may have trouble organizing their thoughts and speech. Sometimes a person will stop talking in the middle of a thought, jump from topic to topic, or make up words that have no meaning.
Movement disorder: When a person exhibits abnormal body movements. People with movement disorder may repeat certain motions over and over.

Negative symptoms include loss of motivation, loss of interest or enjoyment in daily activities, withdrawal from social life, difficulty showing emotions, and difficulty functioning normally. Negative symptoms include:
Having trouble planning and sticking with activities, such as grocery shopping.
Having trouble anticipating and feeling pleasure in everyday life.
Talking in a dull voice and showing limited facial expression.
Avoiding social interaction or interacting in socially awkward ways.
Having very low energy and spending a lot of time in passive activities. In extreme cases, a person might stop moving or talking for a while, which is a rare condition called catatonia.

These negative symptoms are sometimes mistaken for symptoms of depression or other mental illnesses. The National Institute of Mental Health Measurement and Treatment Research to Improve Cognition in Schizophrenia consensus panel has defined five negative symptoms (Kirkpatrick et al., 2006, The NIMH-MATRICES Consensus Statement on Negative Symptoms, Schizophrenia Bulletin 32 (2): 214-29): blunted affect (diminished facial and emotional expression), alogia (decrease in verbal output or verbal expressiveness), asociality (lack of involvement in social relationships of various kinds), avolition (a subjective reduction in interests, desires, and goals and a behavioral reduction of self-initiated and purposeful acts), and anhedonia (inability to experience pleasure from positive stimuli). The current DSM-5 (American Psychiatric Association. Diagnostic and Statistical Manual of Mental Disorders. 5th ed. Washington D.C: American Psychiatric Association; 2013) describes negative symptoms as “restricted emotional expression and avolition”. The first term includes reduction in expressions of emotion “in the face, eye contact, intonation of speech (prosody), and movements of the hand, head, and face that normally give an emotional emphasis to speech”. Avolition has been defined as “a decrease in motivated self-initiated purposeful activities”. Certain other negative symptoms have been mentioned and defined in the current system and include alogia, anhedonia, and asociality.

Cognitive symptoms include problems in attention, concentration, and memory. These cognitive symptoms can make it hard to follow a conversation, learn new things, or remember appointments. A person's level of cognitive functioning is one of the best predictors of their day-to-day functioning. Cognitive symptoms include:
Having trouble processing information to make decisions.
Having trouble using information immediately after learning it.
Having trouble focusing or paying attention.

Many subjects suffering from schizophrenia emphasize that, in particular, the negative symptoms are problematic in the everyday life. There is therefore a need to treat such negative symptoms in subjects suffering from schizophrenia. The treatment should, however, not negatively affect or worsen the positive symptoms or the cognitive symptoms of schizophrenia. The dopaminergic hypothesis of schizophrenia proposes that the positive symptoms are the result of hyperactivity of dopaminergic neurotransmission in dorsal striatal pathways, while the negative symptoms are thought to arise from hypodopaminergic functioning in the frontal lobe and additional dorsal and mesolimbic structures. Hence, increased dopamine (DA) levels would be beneficial to treat the negative symptoms of schizophrenia. However, such increased DA levels would in fact aggravate the positive symptoms of schizophrenia, which is not desired.

The ventral striatum is a crucial region in the reward-circuitry and due to its role in experiences of reward and pleasure. Especially, it has been suggested to play an important role in anhedonia and depressive-like negative symptoms of schizophrenia. The ventral striatum is, thus, a tentative target for treatment of these symptoms, and given a presumed hypodopaminergic state in this region, an ideal treatment would selectively increase ventral striatum DA transmission without interfering with dorsal striatum (DS) DA, thereby avoiding a worsening of the positive symptoms. Striatal DA is regulated by two of the main dopaminergic systems, namely the mesolimbic and the nigrostriatal pathways. The mesolimbic pathway originates in ventral tegmental area (VTA) where DA neurons project to the nucleus accumbens (nAc) in the ventral striatum, whereas the nigrostriatal pathway runs from the substantia nigra pars compacta (SNc) to the dorsal striatum, which in humans comprises the caudate nucleus and putamen. This arrangement theoretically offers a possibility to selectively increase DA in the nAc, without causing a simultaneous elevation in DS.

Drugs with a high psychosis generating potential, such as amphetamine and cocaine, produce a substantial DA elevation in both the nAc and the dorsomedial striatum (DMS, analogous to the associative striatum in humans). However, as shown in the Example section, nicotine, a drug with essentially no psychosis generating properties, elevates DA in the nAc only. This differential effect on striatal DA might be due to the different mechanisms by which these drugs affect DA release. Cocaine and amphetamine act on the dopaminergic terminals, by interfering with DA transporters. Nicotine, on the other hand, acts on nicotinic acetylcholine receptors (nAChRs), most likely located on DA neurons and on glutamatergic terminals in the VTA, thereby increasing neuronal firing and promoting DA release primarily in the nAc. Ethanol, another addictive substance with low psychosis generating capabilities, affects striatal DA in markedly different ways, with a slower and less potent DA increase in DMS, as compared to the nAc. Ethanol is believed to elevate DA in the nAc by interfering with glycine receptors in nAc, which secondarily leads to indirect activation of specific sub-types of nAChRs in the anterior VTA. Ethanol is an allosteric agonist of glycine receptors.

Experimental data as presented herein shows that the combination of ethanol and nicotine indeed raises nAc DA but not DMS DA. A similar effect of enhanced nAc DA release but no significant enhancement of DMS DA release was seen for the combination of the partial nAChR receptor agonist varenicline and the glycine transport 1 (GlyT1) inhibitor Org 24598. The combination of varenicline and Org 24598 leads to a significant increase in nAc DA release without any enhancement of DA release in DMS. In fact, varenicline alone resulted in a significant DA increase in both nAc and, to a lesser extent, also in DMS but when combined with Org 24598, which alone caused a minor DA increase in both nAc and DMS, the combination resulted in an even higher DA increase in nAc but a reduction of DA increase in DMS as compared to varenicline alone. This effect was highly surprising as both compounds alone resulted in a DA increase in both nAc and DMS but when combined Org 24598 potentiated the varenicline-induced DA increase in nAc but suppressed the varenicline-induced DA increase in DMS. The combination thereby resulted in a significant DA increase in nAc but no significant increase in DMS. Hence, a selective increase in DA in the nucleus accumbens (nAc, part of the ventral striatum) is achieved without altering DA levels in the DMS. Further, the GlyT2 inhibitor Org 25543 was able to increase nAc DA levels following systemic administration but at the same time lower DMS DA levels.

An aspect of the invention thereby relates to a combination comprising an effective amount of varenicline, or a salt thereof, and an effective amount of a glycine transport (GlyT) inhibitor. This combination achieves a selective increase in DA in the ventral striatum, and in particular in the nucleus accumbens (nAc) of the ventral striatum, but no accompanying significant increase in DA in the dorsal striatum, and in particular in the associative striatum of the dorsal striatum.

As used herein, “varenicline”, 7,8,9,10-tetrahydro-6,10-methano-6H-pyrazino[2,3-h][3]benzazepine, see formula I below, includes the originator drug substance and modified derivatives thereof.

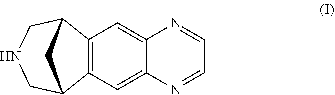 [SMILES: c1cnc2cc3c(cc2n1)[C@@H]1CNC[C@H]3C1]

Varenicline is commercially available under the name CHANTIX® or CHAMPIX® in the form of a tartrate salt of varenicline, i.e., varenicline tartrate, (2R,3R)-2,3-dihydroxysuccinic acid-5,8,14-triazatetracyclo[10.3.1.0^{2,11}.0^{4,9}]hexadeca-2,4 (9),5,7,10-pentaene (1:1), see formula II below.

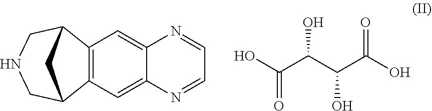 [SMILES: c1cnc2cc3c(cc2n1)[C@@H]1CNC[C@H]3C1 ; O=C(O)[C@H](O)[C@@H](O)C(=O)O]

Varenicline and its effective use in smoking cessation is well known. It is available on prescription and acts as a nicotinic receptor partial agonist and therefore reduces cravings for and decreases the pleasurable effects of cigarettes and other tobacco products.

A currently preferred salt of varenicline is varenicline tartrate.

As used herein, “glycine transport (GlyT) inhibitor” is an agent, compound or drug that inhibits the reuptake of the neurotransmitter glycine by blocking one or more of the glycine transporters. GlyT inhibitor is also referred to as glycine reuptake inhibitor (GRI) in the art. In humans, there are two different glycine transporters; glycine transporter 1, also referred to as sodium- and chloride-dependent glycine transporter 1 encoded by the SLC6A9 gene, and glycine transporter 2, also referred to as sodium- and chloride-dependent glycine transporter 2 encoded by the SLC6A5 gene.

In an embodiment, the GlyT inhibitor is a GlyT1 inhibitor, i.e., an inhibitor that selectively inhibits GlyT1 but does not substantially affect GlyT2. Examples of selective GlyT1 inhibitors include bitopertin ({4-[3-fluoro-5-(trifluoromethyl)pyridin-2-yl]piperazin-1-yl}{5-(methylsulfonyl)-2-[(1S)-2,2,2-trifluoro-1-methylethoxy]phenyl}methanone), Org 24598 (N-methyl-N-[(3R)-3-phenyl-3-[4-(trifluoromethyl) phenoxy]propyl]-glycine), Org 25935 (2-([(1R,2S)-6-methoxy-1-phenyl-1,2,3,4-tetrahydronaphthalen-2-yl]methyl-methylamino) acetic acid), ALX-5407 (2-[[(3R)-3-(4-fluorophenyl)-3-(4-phenylphenoxy) propyl]-methylamino]acetic acid), ASP2535 (4-[3-isopropyl-5-(6-phenyl-3-pyridyl)-4H-1,2,4-triazol-4-yl]-2,1,3-benzoxadiazole), BI 425809 (iclepertin, CAS No.: 1421936-85-7), pesampator (N-[(3S,4S)-4-[4-(5-cyanothiophen-2-yl) phenoxy]oxolan-3-yl]propane-2-sulfonamide), PF-03463275 (N-[(3-chloro-4-fluorophenyl)methyl]-1-methyl-N-[(1R,5S)-3-methyl-3-azabicyclo[3.1.0]hexan-6-yl]methyl]imidazole-4-carboxamide), tilapertin ((R)-2-(4-(phenyl(3-(trifluoromethyl)phenyl)methyl) piperazin-1-yl) acetic acid) and sarcosine ((methylamino) acetic acid).

In another embodiment, the GlyT inhibitor is a GlyT2 inhibitor, i.e., an inhibitor that selectively inhibits GlyT2 but does not substantially affect GlyT1. Examples of selective GlyT2 inhibitors include Org 25543 (N-[1-(dimethylamino)cyclopentyl]methyl]-3,5-dimethoxy-4-(phenylmethoxy)benzamide hydrochloride), opiranserin (4-butoxy-N-([4-(dimethylamino) oxan-4-yl]methyl)-3,5-dimethoxybenzamide), VVZ-368 (active metabolite of opiranserin), and N-arachidonylglycine ([(5Z,8Z,11Z,14Z)-Icosa-5,8,11,14-tetraenamido]acetic acid).

Further examples of GlyT inhibitors are disclosed in the following patents publications: WO 2010/092181 disclosing heterocyclic sulphonamide compounds that inhibit GlyT1; WO 2010/092180 disclosing aminotetraline derivatives that inhibit GlyT1; WO 2011/153359 disclosing GlyT1 inhibitors; WO 2008/002583 disclosing compounds that inhibit GlyT1; WO 2010/087762 disclosing 2-aza-bicyclo[2.2.1]heptane compounds that modulate GlyT1; WO 2013/017657 disclosing phenyl-3-aza-bicyclo[3.1.0]hex-3-yl-methanones that inhibit GlyT1; WO 2008/092878 disclosing GlyT1 inhibitors; WO 2012/019970 disclosing a combination of a GlyT1 inhibitor and an atypical antipsychotic drug; WO 2010/086251 disclosing aroylamino- and heteroaryoylamino-substituted piperidines as GlyT1 inhibitors; WO 2011/161006A1 disclosing amido-tropane derivatives that are good inhibitors of GlyT1 and have good selectivity for GlyT2; WO 2011/161008 disclosing quinolizidine and indolizidine derivatives that are good inhibitors of GlyT1 and have good selectivity for GlyT2; WO 2011/095434 disclosing tetrahydro-pyran derivatives that are good inhibitors of GlyT1; WO 2006/072436 disclosing [4-(heteroaryl) piperazin-1-yl)-2 (2,5-substituted-phenyl) methanone derivatives as GlyT1 inhibitors; WO 2006/077026 disclosing 2,5-disubstituted phenyl methanone derivatives as GlyT1 inhibitors; WO 2012/036278 disclosing GlyT inhibitors; WO 2012/036276 disclosing GlyT inhibitors; WO 2012/036268 disclosing GlyT inhibitors; WO 2010/107115 disclosing GlyT inhibitors; WO 2006/110724 disclosing GlyT1 inhibitors; WO 2006/039221A3 disclosing cyclopropyl piperidine GlyT inhibitors; WO 2013/187503 disclosing GlyT inhibitors; and WO 2011/007899 disclosing GlyT inhibitors, the teachings of which with regard to GlyT inhibitors is hereby incorporated by reference as examples of GlyT inhibitors that can be used according to the invention.

In an embodiment, the combination comprises an effective amount of varenicline, or the salt thereof, and an effective amount of one GlyT inhibitor. In another embodiment, the combination comprises an effective amount of varenicline, or the salt thereof, and a respective effective amount of multiple, i.e., at least two, different GlyT inhibitors.

In an embodiment, the combination comprises an effective amount of varenicline, or the salt thereof, and an effective amount of one GlyT1 inhibitor. In another embodiment, the combination comprises an effective amount of varenicline, or the salt thereof, and an effective amount of one GlyT2 inhibitor. In a further embodiment, the combination comprises an effective amount of varenicline, or the salt thereof, and an effective amount of multiple GlyT1 inhibitors. In yet another embodiment, the combination comprises an effective amount of varenicline, or the salt thereof, and an effective amount of multiple GlyT2 inhibitors. In a further embodiment, the combination comprises an effective amount of varenicline, or the salt thereof, and an effective amount of at least one GlyT1 inhibitor and an effective amount of at least one GlyT2 inhibitor.

In a particular embodiment, the GlyT inhibitor is N-methyl-N-[(3R)-3-phenyl-3-[4-(trifluoromethyl) phenoxy]propyl]-glycine (Org 24598).

In another particular embodiment, the GlyT inhibitor is N-[1-(dimethylamino)cyclopentyl]methyl]-3,5-dimethoxy-4-(phenylmethoxy)benzamide hydrochloride (Org 25543).

The compounds (i.e., varenicline, or the salt thereof, and/or GlyT inhibitor), combinations and/or compositions described herein are for administration in an effective amount. An “effective amount” (or “therapeutically effective amount”) is an amount that alone, or together with further doses, produces the desired (therapeutic) response. The (therapeutically) effective amount to be used will depend, for example, upon the therapeutic objectives, the route of administration, and the condition of the subject. A suitable dosage of varenicline, or the salt thereof, and/or the GlyT inhibitor for a given subject can be determined by an attending physician, taking into consideration various factors known to modify the action of drugs including severity and type of disease, body weight, sex, diet, time and route of administration, other medications and other relevant clinical factors. Accordingly, in one example, a suitable dose of varenicline, or the salt thereof, and/or the GlyT inhibitor is selected based on the body weight of the subject. The dosages and schedules may be varied according to the particular disease state and the overall condition of the subject. Suitable doses may also be determined based on the severity of the subject. Suitable doses may also be determined for subgroups of subjects, e.g., based on their heredity and/or pharmocogenetic profile(s).

In an embodiment, the effective amount of varenicline, or the salt thereof, is selected within a range of from 0.1 mg/day to 5 mg/day. For instance, the effective amount of varenicline, or the salt thereof, is selected within a range of from 0.5 mg/day to 5 mg/day, such as from 0.5 mg/day to 2 mg/day. The effective amount may differ depending on the stage of treatment. For example, for methods of treatment that include a variable dose regimen, different doses may be effective during the induction phase(s) and the treatment (or maintenance) phase. By way of example, a first induction phase may use an initial dose of varenicline, or the salt thereof, of about 0.5 mg/day. Accordingly, about 0.5 mg/day may be considered an effective amount of varenicline, or the salt thereof, for use during the first induction phase. Subsequently, a second induction phase may use an increased dose of varenicline, or the salt thereof, of about 1 mg/day. Accordingly, about 1 mg/day may be considered an effective amount of varenicline, or the salt thereof, for use during the second induction phase. Finally, a maintenance or treatment phase may use a dose of varenicline, or the salt thereof, of about 2 mg/day or up to about 5 mg/day. Accordingly, about 2 mg/day or up to about 5 mg/day may be considered an effective amount of varenicline, ore the salt thereof, for use during the treatment phase.

Correspondingly, the effective amount of the GlyT inhibitor is preferably at least partly dependent on the particular type of GlyT inhibitor used. For instance, BI 425809 could be administered in an amount of from 1 mg up 150 mg, preferably from 2 mg up to 100 mg, such as from 10 mg up to 75 mg once or twice daily, PF-03463275 could be administered in an amount of from 1 mg up to 100 mg, preferably from 10 mg up to 60 mg once or twice daily, bitopertin could be administered in an amount of from 1 mg up to 50 mg, preferably from 5 mg up to 20 mg once or twice daily, tilapertin could be administered in an amount of from 1 mg up to 100 mg, preferably from 5 mg up to 40 mg once or twice daily, and Org 25935 could be administered in an amount of from 1 mg up to 50 mg, preferably from 10 up to 15 mg once or twice daily. Hence, in a general embodiment, the effective amount of the GlyT inhibitor is preferably from about 1 mg up to about 250 mg once or multiple times, such as twice, daily, preferably from about 5 mg up to about 150 mg once or multiple times, such as twice, daily.

As used herein, a “combination” comprising varenicline, or the salt thereof, and the GlyT inhibitor encompasses a dosage form of varenicline, or the salt thereof, for use in combination with a distinct dosage form of the GlyT inhibitor, as well as a dosage form comprising both varenicline, or the salt thereof, and the GlyT inhibitor. “Combined use” and “combination” in the context of the invention therefore also includes a product comprising both varenicline, or the salt thereof, and the GlyT inhibitor, as discrete separate dosage forms, in separate containers or e.g., in blisters containing both types of drugs in discrete solid dosage units, e.g., in a form in which the dosage units which have to be taken together or which have to be taken within one day are grouped together in a manner which is convenient for the subject. The product itself or as a part of a kit may contain instructions for the simultaneous, sequential or separate administration of the discrete separate dosage units, to a subject. Accordingly, the product may comprise varenicline, or the salt thereof, and the GlyT inhibitor as discrete separate dosage forms, in a form which is suitable for sequential, separate and/or simultaneous administration. The compounds, combinations and/or compositions may be provided in a form, which is suitable for sequential (consecutive), separate and/or simultaneous (concurrent) administration to the subject, in any order. For example, varenicline, or the salt thereof, may be provided in a form that is suitable for sequential, separate and/or simultaneous administration to the GlyT inhibitor. Accordingly, varenicline, or the salt thereof, may be administered to the subject at the same time or at a different time (before or after) compared to when the GlyT inhibitor is administered. In cases where varenicline, of the salt thereof, and the GlyT inhibitor are administered simultaneously, the varenicline, or the salt thereof, and the GlyT inhibitor may be administered as separate compositions that are administered at the same time, or may be administered as a combined composition that includes both varenicline, or the salt thereof, and the GlyT inhibitor.

Hence, in an embodiment, varenicline, or the salt thereof, and the GlyT inhibitor are formulated for sequential administration.

In another embodiment, varenicline, or the salt thereof, and the GlyT inhibitor are formulated for simultaneous administration.

Varenicline, or the salt thereof, and the GlyT inhibitor may formulated for separate administration or for combined administration.

The compounds, combinations and/or compositions described herein can be administered to the subject by any conventional route, including oral administration, for example in tablet form, injection or by gradual infusion over time. The administration may, for example, be topical, oral, parenteral, intravenous, intraperitoneal, intramuscular, intravascular, intracavity, intranasal, intracerebral, intratracheal, intralesional, intraperitoneal, rectal, subcutaneous, transdermal, epidural, percutaneous, or by infusion. By way of example, varenicline, or the salt thereof, can be administered orally, e.g., in tablet form or as a chewing gum composition such as disclosed in WO 2006/100595, transdermal such as disclosed in WO 2007/012963, via controlled release such as disclosed in WO 2009/034431, or may be formulated for intranasal, buccal, sublingual and pulmonary delivery, such as disclosed in WO 2006/040680. In an embodiment, varenicline, or the salt thereof, and the GlyT transporter are provided in distinct compositions that are suitable for e.g., sublingual administration, administration by nasal spray, implantation, and or administration by pump.

The compounds, combinations and/or compositions described herein may therefore be in a form suitable for the above modes of administration. For example, suitable forms for oral administration include a tablet or capsule; suitable forms for nasal administration or administration by inhalation include a powder or solution; suitable forms for parenteral injection, including intravenous, subcutaneous, intramuscular, intravascular or infusion, include a sterile solution, suspension or emulsion; suitable forms for topical administration include a patch, an ointment or cream; and suitable forms for rectal administration include a suppository. Alternatively, the route of administration may be by injection.

The compounds, combinations and/or compositions of the present invention are advantageously presented in unit dosage form. Dosage forms (also called unit doses) are pharmaceutical drug products in the form, in which they are marketed for use, with a specific mixture of active ingredients and inactive components (excipients), in a particular configuration, such as a capsule shell, and apportioned into a particular dose. Depending on the route of administration, dosage forms include liquid, solid, and semisolid dosage forms. Common dosage forms include pills, tablets, capsules, drinks or syrups.

In an example, the combination of varenicline, or the salt thereof, and the GlyT inhibitor is provided in the form of a depot preparation, wherein varenicline, or the salt thereof, and the GlyT inhibitor have be formulated to have the same or different release rates. As used herein, a “depot preparation” refers to a specific formulation of varenicline, or the salt thereof, and the GlyT inhibitor that is given by injection, wherein the medication is slowly released into the body of the subject over a number of days or weeks.

In an example, an effective dose of varenicline, or the salt thereof, may be combined with the corresponding effective dose of the GlyT inhibitor in a unit dosage form, e.g., a tablet, for daily oral administration by the subject. Where the administration of the separate formulations of varenicline, or the salt thereof, and the GlyT inhibitor is sequential or separate, the delay in administering the second formulation should not be such as to lose the beneficial effect of the combination therapy.

Varenicline, or the salt thereof, and/or the GlyT inhibitor may be part of a composition, such as a pharmaceutical composition, that comprises the compound, i.e., varenicline, or the salt thereof, and/or the GlyT inhibitor, and one or more other components. A composition may be a pharmaceutical composition that comprises varenicline, or the salt thereof, and/or the GlyT inhibitor and a pharmaceutically acceptable excipient, adjuvant, diluent and/or carrier. Pharmaceutical compositions may routinely contain pharmaceutically acceptable concentrations of salt, buffering agents, preservatives, compatible carriers, supplementary immune potentiating agents, such as adjuvants and cytokines and optionally other therapeutic agents or compounds.

As used herein, “pharmaceutically acceptable” refers to a material that is not biologically or otherwise undesirable, i.e., the material may be administered to an individual along with the selected compound without causing any undesirable biological effects or interacting in a deleterious manner with any of the other components of the pharmaceutical composition in which it is contained.

Excipients are natural or synthetic substances formulated alongside an active ingredient, i.e., varenicline, or the salt thereof, and/or the GlyT inhibitor, included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned, such as by facilitating powder flowability or non-stick properties, in addition to aiding in vitro stability, such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, magnesium stearate, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art. Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

The compounds, compositions and/or combinations of the invention may be administered using any suitable method and dosage form, as described in detail above.

By way of example, varenicline, or the salt thereof, may be administered to a subject in need thereof using a variable dosage or variable dose regimen comprising at least one induction dose, with a subsequent treatment dose (also known as a maintenance dose).

Appropriate variable dosages for administration of varenicline, or the salt thereof, to humans are well known. As one example, varenicline, or the salt thereof, may be administered in the following doses: first induction dose (days 1 to 3) of 0.5 mg/day; second induction dose (days 4 to 7) of 1 mg/day (e.g., in 2×0.5 mg dosage forms); maintenance dose (from day 8 to 98) of 2 mg/day (e.g., in 4×0.5 mg dosage forms). This example treatment regimen is for a total of 14 weeks, including 12 weeks of steady state treatment.

The daily induction and/or maintenance dose of varenicline, or the salt thereof, may be administered as a single dose, or as a series of doses. In the latter case, the series of doses are to be taken simultaneously, or are taken sequentially, for instance spread out during the day. For example, if the daily second induction dose of varenicline, or the salt thereof, is 1 mg/day, this can either be administered as 1 mg once a day, e.g., two tablets of 0.5 mg taken simultaneously, or as 0.5 mg twice a day, e.g., one tablet of 0.5 mg in the morning and one tablet of 0.5 mg in the evening.

Although a specific variable dose regimen for varenicline, or the salt thereof, is provided above, the invention is not limited to this variable dose regimen. The specific dosages, number of days for each dose and the concentration of each dosage form may vary depending on several factors such as severity of disease, sex, weight, age etc. of the subject. Identifying appropriate dose regimens for the administration of varenicline in subjects in need thereof is within the routine capabilities of a person of skill in the art.

By way of a further example, the GlyT inhibitor may be administered to a subject in need thereof using a variable dose regimen or variable dosage. In such a case, the variable dosage comprises at least one induction dose, with a subsequent treatment dose (also known as a maintenance dose). Appropriate variable dose methods for administration of the GlyT inhibitor could be used. For instance a first induction dose could be used for, for instance, days 1 to 7 followed by a maintenance dose, such as from days onwards, such as up to day 98. In an embodiment, the daily maintenance dose is higher than the daily first induction dose, such as twice the daily induction dose. This example treatment regimen is for a total of 14 weeks, including 12 weeks of steady state treatment. As described above in respect of varenicline, the daily induction and/or maintenance dose of the GlyT inhibitor may be administered as a single dose, or as a series of doses. In the latter case, the series of doses are to be taken simultaneously, or are taken sequentially e.g., spread out during the day.

Although a specific variable dose regimen for the GlyT inhibitor is provided above, the invention is not limited to this regimen; the specific dosages, number of days for each dose and the concentration of each dosage form may vary depending on several factors such as severity of the disease, sex, weight, age etc. of the subject. Identifying appropriate dose regimens for the administration of the GlyT inhibitor is within the routine capabilities of a person of skill in the art. For the avoidance of doubt, the invention is directed to the combined use of varenicline, or the salt thereof, and the GlyT inhibitor and, thus, the invention may be achieved by combining the two specific variable dose regimens for varenicline, or the salt thereof, and the GlyT inhibitor provided above. As mentioned previously, the dosage forms for varenicline, or the salt thereof, and the GlyT inhibitor may be suitable for separate, simultaneous or sequential administration.

Preferably, the daily dose amounts of varenicline, or the salt thereof, and/or the GlyT inhibitor are administered to the subject as a single dose, or alternatively are administered as multiple doses. As used herein, the term “dose” refers to an amount of the drug (varenicline, or the salt thereof, or the GlyT inhibitor), which is administered to the subject.

As used herein, the term “variable dose” includes different doses of varenicline, or the salt thereof, and/or the GlyT inhibitor, which are administered to a subject for therapeutic treatment. “Variable dose regimen” or “variable dosage” describes a treatment schedule that is based on administering different amounts of varenicline, or the salt thereof, and/or the GlyT inhibitor at various time points throughout the course of treatment. In an embodiment, the invention describes a variable dose comprising an induction phase and a treatment phase, wherein varenicline, or the salt thereof, and/or the GlyT inhibitor is administered at a lower dose during the induction phase than the treatment phase. As used herein, the terms “maintenance phase” and “treatment phase” are used interchangeably. Similarly, “maintenance dose” and “treatment dose” are used interchangeably. The term “induction phase” refers to a period of treatment comprising administering of varenicline, or the salt thereof, and/or the GlyT inhibitor to a subject in order to attain a threshold level. During the induction phase, at least one induction dose of the varenicline, or the salt thereof, and/or the GlyT inhibitor is administered to a subject. The induction phase may vary in length, e.g., at least 1, 2, 3, 4, 5, 6, 7, days. Typically, the induction phase is 5 to 8 days, such as 7 days. The induction phase may be split into e.g., a first induction phase and a second induction phase if more than one induction dose is to be used. In this case, the first induction phase may be about 1 to 3 days, and the second induction phase may subsequently be an additional 1 to 4 days, e.g., first induction phase-day 1 to 3, second induction phase-day 4 to 7, treatment phase-day 8 onwards.

The term “threshold level” refers to therapeutically effective level of varenicline, or the salt thereof, and/or the GlyT inhibitor in a subject. A threshold level is achieved by administering at least one induction dose during the induction phase of treatment. Any number of induction doses may be administered to achieve a threshold level of varenicline, or the salt thereof, and/or the GlyT inhibitor. Once a threshold level is achieved, the treatment phase is initiated.

The term “induction dose” refers to the first dose of varenicline, or the salt thereof, and/or the GlyT inhibitor, which is smaller in comparison to the maintenance or treatment dose. The induction dose can be a single dose or alternatively, a set of doses.

The induction dose is often used to bring the drug in the body to a steady state amount. The induction dose is administered during the induction phase of the therapy. The induction dose may be increased during the induction phase. For example, the induction phase for varenicline administration may be split into a first induction phase, wherein a first induction dose of e.g., 0.5 mg varenicline/day is administered, and a second induction phase, wherein a second induction dose of e.g., 1 mg/day is administered. Alternatively, the induction dose may remain constant during the induction phase.

The “treatment phase” or “maintenance phase” refers to a period of treatment comprising administration of varenicline, or the salt thereof, and/or the GlyT inhibitor to a subject in order to achieve and maintain a desired therapeutic effect. The treatment phase follows the induction phase, and therefore is initiated once a threshold level is achieved.

The term “treatment dose” or “maintenance dose” is the amount of varenicline, or the salt thereof, and/or the GlyT inhibitor taken by a subject to achieve and maintain a desired therapeutic effect. A treatment dose is administered subsequent to the induction dose. A treatment dose can be a single dose, or alternatively, a set of doses. A treatment dose is administered during the treatment phase of the therapy. Treatment doses are higher than the induction dose and can be equal to each other when administered in succession. Typically, the maintenance dose remains constant throughout the treatment phase of the therapy (e.g., about 2 mg/day for varenicline, or the salt thereof,). However, the maintenance dose may also be varied during the treatment phase, provided that the desired therapeutic effect is maintained.

The treatment phase of the therapy may be of any appropriate length. By way of example, the treatment phase may be at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks.

The combination of varenicline, or the salt thereof, and the GlyT transporter of the invention achieves a selective increase in DA levels when administered to a subject. In particular, the combination of varenicline, or the salt thereof, and the GlyT transporter selectively elevated ventral striatum DA (nAc DA) whilst not significantly altering dorsal striatum DA (associative striatum DA). Such a selective DA elevation is of benefit for subjects suffering from schizophrenia. In particular, the combination of varenicline, or the salt thereof, and the GlyT transporter is useful for treatment of the negative symptoms in schizophrenia, without the risk of exacerbating the positive symptoms.

Treatment of negative symptoms of schizophrenia include treatment of one or more, such as all, of the negative symptom selected from the group consisting of blunted, alogia, asociality, avolition and anhedonia.

The treatment of such negative symptoms of schizophrenia preferably does not exacerbate positive symptoms of schizophrenia. Such positive symptoms include hallucinations, delusions, thought disorder and movement disorder.

Another aspect of the invention relates to a combination according to the invention for use as a medicament.

A particular aspect of the invention relates to a combination according to the invention for use in treatment of schizophrenia. A related aspect defines use of a combination according to the invention for the manufacture of a medicament for treatment of schizophrenia.

Another particular aspect of the invention relates to a combination according to the invention for use in treatment of negative symptoms of schizophrenia in a subject suffering from schizophrenia. A related aspect defines use of a combination according to the invention for the manufacture of a medicament for treatment of negative symptoms of schizophrenia in a subject suffering from schizophrenia.

In a particular embodiment, the combination does not exacerbate positive symptoms of schizophrenia.

The present invention also relates to a method for treatment of schizophrenia. The method comprises administering a combination according to the invention to a subject suffering from schizophrenia. The present invention also relates to a method for treatment of negative symptoms of schizophrenia. The method comprises administering a combination according to the invention to a subject suffering from schizophrenia.

As used herein, the terms “treat”, “treating” and “treatment” are taken to include an intervention performed with the intention of preventing the development or altering the pathology of a disorder or symptom.

Accordingly, “treatment” refers to both therapeutic treatment and prophylactic or preventative measures, wherein the object is to prevent or slow down (lessen) the targeted disorder or symptom. Accordingly, the term “treating” encompasses treating and/or preventing the development of a disorder or symptom. The invention may therefore be useful for preventing schizophrenia and/or negative symptoms of schizophrenia in a subject that is susceptible to developing schizophrenia. The invention may be a preventative treatment of relapse in schizophrenia and/or negative symptoms of schizophrenia. Treatment as used herein also include inhibition of the disease, including symptoms thereof.

As used here in the term “subject” refers to an individual, e.g., a human, suffering from or at risk of suffering from, i.e., susceptible to developing, schizophrenia and/or negative symptoms of schizophrenia. The subject may be a patient, i.e., a subject in need of treatment in accordance with the invention. The subject may have received treatment for the disorder or symptom. Alternatively, the subject has not been treated prior to treatment in accordance with the present invention.

### EXAMPLE

### Example 1

Negative symptoms in schizophrenia are largely resistant to available treatments. Studies have indicated that negative symptomatology is, at least in part, connected to a hypodopaminergic state in the ventral striatum, an important part of the reward circuitry of the brain. Elevating ventral striatum dopamine (DA) is complicated by the risk of increasing positive symptoms, an effect putatively associated with the dorsomedial striatum (DMS). In this Example, we aimed to selectively increase DA in the nucleus accumbens (nAc, part of the ventral striatum) without altering DA levels in the DMS. To this end, we employed microdialysis and targeted nicotinergic acetylcholine receptors (nAChR) using nicotine, and the partial nAChR agonist varenicline, and glycine signalling using ethanol and the glycine reuptake inhibitor Org 24598, in a Wistar rat model. The first set of experiments, where ethanol and nicotine were used alone and in combination, was carried out using a single probe microdialysis setup. The second set of experiments, using Org 24598 and varenicline alone and in combination, utilised a dual microdialysis probe that allows for simultaneous sampling in nAc and DMS. The single probe experiments showed that a combination of ethanol and nicotine produced an enhanced effect on DA in the nAc but not in the DMS. Similarly, the combination of Org 24598 and varenicline resulted in a potentiated DA release in the nAc, but not in the DMS. The results shows that it is possible to selectively increase ventral striatum DA without risking an excessive DA release in the DMS.

### Methods

### Animals

95 adult male Wistar Han rats (Envigo, Horst, Netherlands) were used in all experiments, weighing 280 to 350 g at the time of surgery (corresponding to approximately 9-10 weeks of age). The animals were housed in groups of five per cage (55×35×20 cm) at constant room temperature (22° C.), relative humidity (65%), and regular dark-light conditions (lights on at 7 AM and off at 7 PM). All animals had ad libitum access to standard rat feed (Harlan Teklad, Envigo, Europe) and tap water. The animals were allowed to acclimate to the animal facilities for at least one week before surgery. This study was approved by the Ethics Committee for Animal Experiments, Gothenburg, Sweden and conducted according to national laws and guidelines for the care and use of laboratory animals.

### Drugs

Nicotine ((−)-nicotine hydrogen tartrate salt, Sigma, Stockholm, Sweden), 95% ethanol (Kemetyl AB, Stockholm, Sweden), varenicline (6,7,8,9-tetrahydro-6,10-methano-6H-pyrazino[2,3-h][3]benzazepine tartrate; provided by Pfizer Global Research and Development) and N-methyl-N-[(3R)-3-phenyl-3-[4-(trifluoromethyl) phenoxy]propyl]-glycine (Org 24598) (Tocris, Bristol, United Kingdom) were dissolved in physiological saline solution (0.9% NaCl) and administered subcutaneous (nicotine and varenicline) or intraperitoneal (ethanol and Org 24598) at a volume of 2 ml/kg (nicotine and varenicline), 5 ml/kg for EtOH and 4 ml/kg for Org 24598. The pH of the nicotine solution was neutralized with bicarbonate to approximately 7.2-7.4. Animals receiving only one active treatment also received a corresponding vehicle injection, to control for the effect of the dual injections.

### Surgery

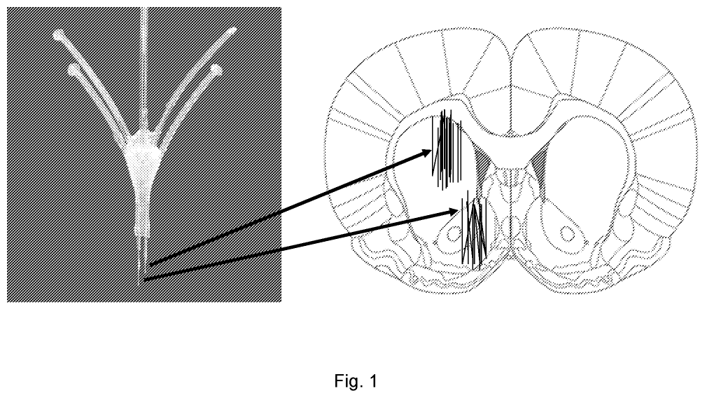

The microdialysis experiments were performed as previously described (Clarke, et al., 2014, Increase in nucleus accumbens dopamine levels following local ethanol administration is not mediated by acetaldehyde, Alcohol Alcohol 49, 498-504; Danielsson et al., 2021, Differential dopamine release by psychosis-generating and non-psychosis-generating addictive substances in the nucleus accumbens and dorsomedial striatum, Transl Psychiatry 11, 472). Animals were anaesthetized with isoflurane before being mounted into a stereotaxic instrument (David Kopf Instruments, AgnTho's, Stockholm, Sweden). The animals were equipped with either a standard in-house made single probe placed in the nAc (A/P +1.85, M/L −1.2, D/V −7.8) or the DMS (A/P+1.2, M/L −1.4, D/V −5.5) (Paxinos, 2007, The rat brain in stereotaxic coordinates. Amsterdam u.a.: Elsevier, Academic Press, Amsterdam), or a custom-made dual microdialysis probe (FIG. 1), which permits simultaneous sampling from both the nAc and DMS. Two additional holes were drilled in the skull in order to allow anchoring screws to be fastened, and the probe and screws were set in place using Harvard cement (DAB Dental AB, Stockholm, Sweden). The animals were then placed in single housing cages and allowed to recover for 48 h prior to the microdialysis experiment.

### Microdialysis

On the day of the experiment, the animals were connected to a microperfusion pump (U-864 Syringe Pump, AgnTho's, Sweden) via a swivel, which allowed the animals to move around the cage freely. During the entire experiment, the probes were perfused with Ringer solution at a rate of 2 μl/min, and dialysate samples were collected every 20 min. Baseline sampling commenced after a 2-hour period, during which a balanced fluid exchange was established. Once at least three consecutive stable DA values were obtained (±10%), pharmacological treatment was administered, followed by a total of 180 minutes of sampling.

### Hplc Analysis of Dopamine

Microdialysis samples were analyzed as previously described for DA (Clarke, et al., 2014, Increase in nucleus accumbens dopamine levels following local ethanol administration is not mediated by acetaldehyde, Alcohol Alcohol 49, 498-504). In brief, DA content was analyzed using high performance liquid chromatography (HPLC) with electrochemical detection, using an external standard containing 3.25 fmol/μl of DA to identify DA peaks and quantify DA content. The chromatograms were analyzed using the software Thermo Scientific Chromelion Chromatography System (CDS) Chromelion 7.

### Verification of Probe Placement

The animals were euthanized immediately following the experiment, their brains removed and fixed in formalin-free fixative (Accustain, Sigma-Aldrich), and stored at 4° C. The brains were later sectioned (approximately 0.5 mm) and probe placement visually verified. Only animals with correct probe placement (FIG. 1) and no signs of bleeding or extraneous damage to the tissue were included in the statistical analysis.

### Statistics

Statistical analysis of microdialysis data was performed using two-way analysis of variance (ANOVA) with repeated measures for the microdialysis data, and ordinary one-way ANOVA with repeated measures followed by a Tukey's post hoc test for the area under the curve (AUC). Both analyses were made for t=0-180 min. A probability (p) value less than 0.05 was considered statistically significant. All values are expressed as mean±SEM. All statistical analyses were made using GraphPad Prism version 8.3.0 for Windows (GraphPad Software, USA).

### Results

Enhanced effects of combined treatment of EtOH and nicotine in the nAc but not in the DMS Single probe microdialysis experiments were conducted to compare the potentially enhanced effects of co-administration of EtOH and nicotine in the nAc and the DMS.

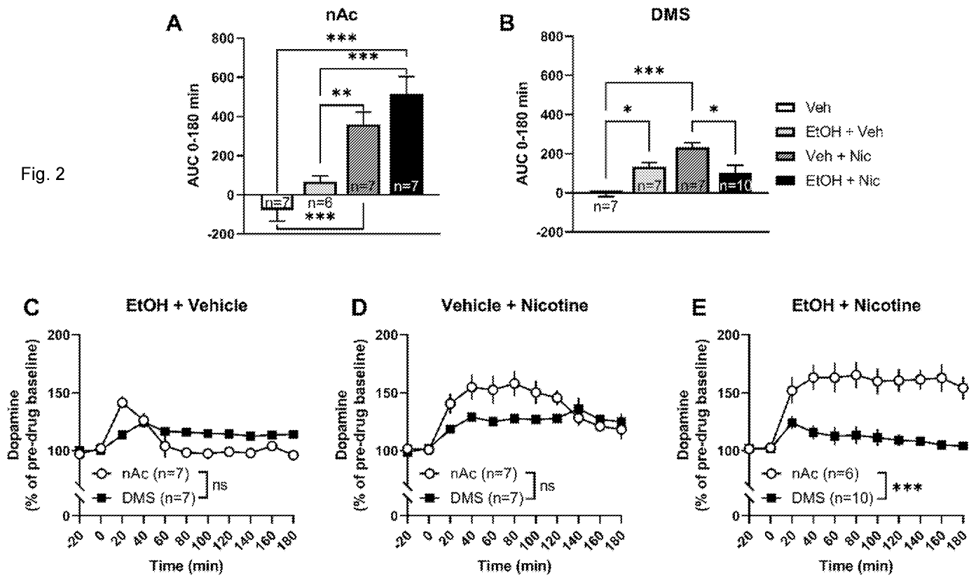

In the nAc, EtOH (2.5 g/kg) produced a transient increase in DA, while nicotine (0.36 mg/kg) in combination with EtOH resulted in a significantly more pronounced elevation of DA as compared to nicotine alone (one-way ANOVA: F_{(3, 23)}=19.82, p<0.001, vehicle+vehicle vs EtOH+vehicle: p=0.32; vehicle+vehicle vs nicotine+vehicle: p<0.001; vehicle+vehicle vs EtOH+nicotine: p<0.001, EtOH+vehicle vs nicotine+vehicle: p=0.009; EtOH+vehicle vs EtOH+nicotine: p<0.001, nicotine+vehicle vs EtOH+nicotine: p=0.30, FIG. 2A).

In the DMS, EtOH significantly increased DA over time but did not potentiate the DA elevation induced by nicotine. Rather EtOH suppressed the DA elevation produced by nicotine alone (one-way ANOVA: F_{(3, 26)}=10.18, p<0.001, vehicle+vehicle vs EtOH+vehicle: p=0.02; vehicle+vehicle vs nicotine+vehicle: p<0.001; vehicle+vehicle vs EtOH+nicotine: p<0.07, EtOH+vehicle vs nicotine+vehicle: p=0.12; EtOH+vehicle vs EtOH+nicotine: p=0.86, nicotine+vehicle vs EtOH+nicotine: p=0.02, FIG. 2B).

When comparing the two brain regions over time, systemic administration of EtOH did not produce statistically different DA elevations in the nAc and the DMS. However, the interaction factor was significant, suggesting that ethanol does affect the nAc and the DMS in a differential manner over time (EtOH+vehicle−nAc vs. DMS, brain region: F_{(1, 12)}=4.10, p=0.07; time: F_{(2.542, 30.50)}=12.89, p<0.001; interaction: F_{(8, 96)}=9.46, p<0.001, FIG. 2C). Similarly, there was no effect by brain region on the DA elevation induced by nicotine (0.36 mg/kg), but the interaction factor was significant (nicotine+vehicle-nAc vs. DMS, brain region: F_{(1, 12)}=3.36, p=0.092; time: F_{(2.757, 33.08)}=4.032, p=0.02; interaction: F_{(9, 96)}=4.44, p<0.001, FIG. 2D). The combination of EtOH and nicotine, however, produced a significantly larger DA elevation in the nAc, when compared to the DMS (EtOH+nicotine-nAc vs. DMS, brain region: F_{(1, 13)}=23.77, p<0.001; time: F_{(3.206, 41.68)}=1.15, p=0.34; interaction: F_{(8, 104)}=1.90, p=0.068, FIG. 2E).

### Enhanced Effect of the Combination of Org 24598 and Varenicline in the nAc but not in DMS

While the initial set of experiments supported the hypothesis and showed that the combination of ethanol and nicotine could selectively increase DA in nAc, this combination of drugs would not be suitable for therapeutical purposes. In the next set of experiments, we, thus, evaluated the effects of the combination of Org 24598 and varenicline. In these experiments, a dual microdialysis system was utilised, allowing for simultaneous sampling in nAc and DMS.

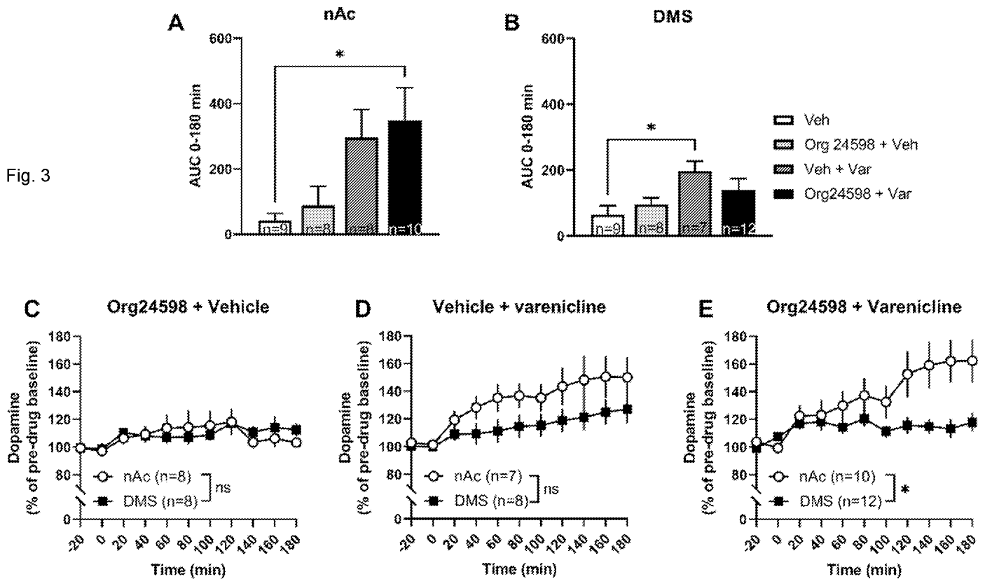

There was no significant effect by the compounds alone when monitoring drug induced DA elevation over time (0-180 min) in the nAc. The combination of Org 24598 (9 mg/kg) and varenicline (1.5 mg/kg), however, significantly elevated nAc DA over time, as compared to vehicle (one-way ANOVA: F_{(3, 31)}=4.25, p=0.013, vehicle+vehicle vs Org 24598+vehicle: p=0.86; vehicle+vehicle vs varenicline+vehicle: p=0.09; vehicle+vehicle vs Org 24598+varenicline: p=0.029, Org 24598+vehicle vs varenicline+vehicle: p=0.19; Org 24598+vehicle vs Org 24598+varenicline: p=0.088, varenicline+vehicle vs Org 24598+varenicline: p=0.86, FIG. 3A).

In the DMS, varenicline alone produced a small but significant elevation of DA over time. This increase, however, was attenuated by the addition of Org 24598 (one-way ANOVA: F_{(3, 32)}=3.06, p=0.042, vehicle+vehicle vs Org 24598+vehicle: p=0.90; vehicle+vehicle vs varenicline+vehicle: p=0.036; vehicle+vehicle vs Org 24598+varenicline: p=0.29, Org 24598+vehicle vs varenicline+vehicle: p=0.17; Org 24598+vehicle vs Org 24598+varenicline: p=0.74, varenicline+vehicle vs Org 24598+varenicline: p=0.55, FIG. 3B).

When comparing the two brain regions over time, the DA elevating response to Org 24598 did not differ between the two areas, or over time (Org 24598+vehicle-nAc vs DMS, brain region: F_{(1, 14)}=0.007, p=0.93; time: F_{(2.486, 34.80)}=1.20, p=0.32; interaction: F_{(8, 112)}=1.34, p=0.23, FIG. 3C). The effect by varenicline on DA did also not differ between brain regions, but there was an effect on DA over time. This effect, however, did not differ significantly between the two groups (varenicline+vehicle-nAc vs DMS, brain region: F_{(1, 13)}=2.70, p=0.12; time: F_{(1.156, 15.03)}=10.61, p=0.004; interaction: F_{(9, 117)}=1.21, p=0.30, FIG. 3D). The combination of Org 24598 and varenicline produced significantly different effects on nAc and DMS DA, with a pronounced difference over time (Org 24598+varenicline-nAc vs DMS, brain region: F_{(1, 20)}=5.09, p=0.04; time: F_{(2.596, 51.92)}=4.65, p=0.008; interaction: F_{(8, 160)}=5.11, p<0.001, FIG. 3E).

Negative symptoms of schizophrenia are difficult to treat and have a substantial negative effect on the patient's well-being and quality of life. It has been theorized that low dopaminergic function in the ventral striatum (nAc in rats) could be a contributing factor to the negative symptoms and, thus, that by elevating ventral striatal DA levels, these symptoms could be alleviated. However, a global increase of DA also poses the risk of exacerbating the positive symptoms, which are thought to be connected to a hyperdopaminergic state in the associative striatum (DMS in rats). Finding a treatment that could increase DA selectively in the ventral striatum could, thus, hold promise for a new treatment for negative symptoms in schizophrenia. The aim of the present Example in rats was therefore to investigate the possibility of selectively elevating nAc DA, without simultaneously significantly altering DMS DA.

To this end, we first used ethanol and nicotine as a proof-of-concept in a single-probe in vivo microdialysis setup. The rationale behind this set of experiment was based in findings that 1) combining ethanol and nicotine produced an enhanced effect on nAc DA and 2) EtOH and nicotine both seemed to have sub-region specific effects on striatal DA, with nicotine especially showing a profound preference for elevating DA in the nAc, over the DMS. In the second set of experiments, we wanted to utilize the mechanisms by which EtOH and nicotine interact with striatal DA but using substances with supposedly lower addictive properties. To this end, we used the partial nAChR agonist and smoking cessation aid varenicline to mimic the effects of nicotine, and the GlyT1 inhibitor Org 24598 to achieve an ethanol-like effect on striatal DA. Encouraging, the data showed that the combination of these drugs produced a substantial increase in DA levels in the nAc, while not significantly affecting DA in the DMS. Indeed, the combination of Org 24598 and varenicline appeared to temper the varenicline-induced DA elevation in the DMS, an effect not seen in the nAc.

Somewhat surprisingly, when assessing AUC there was no significant effect by ethanol on nAc DA, an otherwise very consistent finding. The lack of a significant effect of EtOH alone on nAc DA is, however, likely due to that the transient effect seen when studying the effect over time (FIG. 2C) is diluted when taking a longer than usual time-span into consideration (0-180 min). The combination of EtOH and nicotine had an enhanced effect on nAc DA, and the combination did not have the same effect on DMS DA. Interestingly, both ethanol and Org 24598 instead appeared to suppress the DA elevating properties of nicotine and varenicline, respectively, in the DMS. The effect of nicotine on DA in the nAc is primarily mediated through nicotine receptors in the ventral tegmental area (VTA) and that of ethanol involves glycine receptors in the nAc and nicotinic receptors in the VTA. The mechanisms underlying the DA elevating effects of nicotine and ethanol specifically in the DMS are not fully known but could involve actions both on cell-bodies in the substantia nigra and on the neuronal terminals. It has been shown that glycine receptors are of importance for regulating DA levels in both the dorsal striatum and the nAc, as inhibition of glycine receptors significantly reduces the DA tone. However, it has also been suggested that the DA elevating properties of EtOH involves glycine receptors in the nAc only. The microcircuitry involved in regulating striatal dopaminergic output, thus, appears to differ between the two sub-regions. Which neurotransmitters that are involved is yet to be determined, but the GABAergic influence over DA output appears to be stronger in the nAc, as compared to the DMS. Furthermore, glycine receptors may exert a stronger influence over glutamatergic neurotransmission in the nAc, than in the DMS. It is, thus, possible that the activation of midbrain DA neurons, and release of DA in target regions is modulated by glycine receptor activating substances in a sub-regions specific manner and that this results in dampened DA output in the DMS and potentiated output in the nAc. Since varenicline and Org 24598 were chosen based on that they, at least in part, interact with accumbal DA in a manner similar to nicotine and EtOH, it is possible that the differences in microcircuitry in the nAc and DMS also might affect the interaction between the two drugs in a similar manner. In fact, the differential outcome with respect to nAc and DMS DA after co-administering nicotine and ethanol or varenicline and Org 24598, respectively, gives further support for differential DA release regulation in these two brain regions.

In conclusion, being able to pharmacologically selectively elevate nAc DA whilst not significantly altering DMS DA could potentially lead to a novel way to treat negative symptoms in schizophrenia, without the risk of exacerbating the positive symptoms. In this Example, we showed in the rat that this selectivity can be obtained by combining the GlyT1 inhibitor Org 24598 and the partial nAChR agonist varenicline.

### Example 2

### Methods

### Animals

Adult male Wistar Han rats (Envigo, Horst, Netherlands) were used, weighing approximately 320 g at the time of surgery (corresponding to approximately 9-10 weeks of age). The animals were housed in groups of five per cage (55×35×20 cm) at constant room temperature (22° C.), relative humidity (65%), and regular dark-light conditions (lights on at 7 AM and off at 7 PM). All animals had ad libitum access to standard rat feed (Harlan Teklad, Envigo, Europe) and tap water. The animals were allowed to acclimate to the animal facilities for at least one week before surgery. This study was approved by the Ethics Committee for Animal Experiments, Gothenburg, Sweden and conducted according to national laws and guidelines for the care and use of laboratory animals.

### Surgery

The microdialysis experiments were performed as previously described (Clarke et al., 2014; Danielsson et al., 2021). Animals were anesthetized with isoflurane before being mounted into a stereotaxic instrument (David Kopf Instruments, AgnTho's, Stockholm, Sweden). The animals were equipped with either a standard in-house made single probe placed in the nAc (A/P+1.85, M/L −1.2, D/V −7.8) (Paxinos, 2007)), or a custom-made dual microdialysis probe (FIG. 1), which permits simultaneous sampling from both the nucleus accumbens (nAc) and dorsomedial striatum (DMS). Two additional holes were drilled in the skull in order to allow anchoring screws to be fastened, and the probe and screws were set in place using Harvard cement (DAB Dental AB, Stockholm, Sweden). The animals were then placed in single housing cages and allowed to recover for 48 h prior to the microdialysis experiment.

### Microdialysis

On the day of the experiment, the animals were connected to a microperfusion pump (U-864 Syringe Pump, AgnTho's, Sweden) via a swivel, which allowed the animals to move around the cage freely. During the entire experiment, the probes were perfused with Ringer solution at a rate of 2 μl/min, and dialysate samples were collected every 20 min. Baseline sampling commenced after a 2-hour period, during which a balanced fluid exchange was established. Once at least three consecutive stable dopamine (DA) values were obtained (±10%), pharmacological treatment (4, 8 or 12 mg/kg of the Glyt2-inhibitor Org 25543, i.p.) was administered, followed by a total of 180 minutes of sampling.

### HPLC Analysis of Dopamine

Microdialysis samples were analyzed as previously described for DA (Clarke et al., 2014). In brief, DA content was analyzed using high performance liquid chromatography (HPLC) with electrochemical detection, using an external standard containing 3.25 fmol/μl of DA to identify DA peaks and quantify DA content. The chromatograms were analyzed using the software Thermo Scientific Chromelion Chromatography System (CDS) Chromelion 7.

### Results

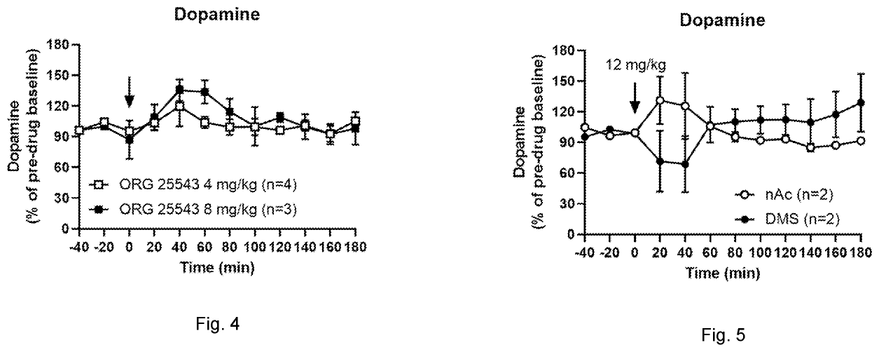

In FIG. 4, the GlyT2 inhibitor Org 25543 displayed a dose-dependent increase of dopamine in the nAc following systemic administration (4 or 8 mg/kg i.p.). The drug was administered at time-point 0 as indicated by the arrow.

In FIG. 5, the GlyT2 inhibitor Org 25543 was administered systemically (12 mg/kg i.p.) and dopamine levels were monitored simultaneously in the aAc and the DSM in the same Wistar rat. Systemic administration of the GlyT2 inhibitor caused an increase in dopamine levels in nAc but at the same time a reduction in dopamine levels in DMS. Hence, the GlyT2 inhibitor Org 25543 causes a selective elevation of nAc DA and a reduction in DMS DA, which is beneficial for treatment of the negative symptoms in schizophrenia without the risk of exacerbating the positive symptoms.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-18. (canceled)

19. A combination comprising an effective amount of varenicline, or a salt thereof, and an effective amount of a glycine transport (GlyT) inhibitor.

20. The combination according to claim 19, wherein varenicline, or the salt thereof, and the GlyT inhibitor are formulated for sequential administration.

21. The combination according to claim 19, wherein varenicline, or the salt thereof, and the GlyT inhibitor are formulated for simultaneous administration.

22. The combination according to claim 19, wherein varenicline, or the salt thereof, and the GlyT inhibitor are formulated for separate administration.

23. The combination according to claim 19, wherein the effective amount of varenicline, or the salt thereof, is selected within a range of from 0.5 mg/day up to 5 mg/day.

24. The combination according to claim 19, wherein the effective amount of the GlyT inhibitor is selected within a range of from 5 mg/day up to 150 mg/day.

25. The combination according to claim 19, wherein the salt of varenicline is varenicline tartrate.

26. The combination according to claim 19, wherein the GlyT inhibitor is a GlyT1 inhibitor.

27. The combination according to claim 26, wherein the GlyT1 inhibitor is selected from the group consisting of {4-[3-fluoro-5-(trifluoromethyl)pyridin-2-yl]piperazin-1-yl}{5-(methylsulfonyl)-2-[(1S)-2,2,2-trifluoro-1-methylethoxy]phenyl}methanone, N-methyl-N-[(3R)-3-phenyl-3-[4-(trifluoromethyl) phenoxy]propyl]-glycine, 2-([(1R,2S)-6-methoxy-1-phenyl-1,2,3,4-tetrahydronaphthalen-2-yl]methyl-methylamino) acetic acid, 2-[(3R)-3-(4-fluorophenyl)-3-(4-phenylphenoxy) propyl]-methylamino]acetic acid, 4-[3-isopropyl-5-(6-phenyl-3-pyridyl)-4H-1,2,4-triazol-4-yl]-2,1,3-benzoxadiazole, BI 425809, N-[(3S,4S)-4-[4-(5-cyanothiophen-2-yl) phenoxy]oxolan-3-yl]propane-2-sulfonamide, N-[(3-chloro-4-fluorophenyl)methyl]-1-methyl-N-[[(1R,5S)-3-methyl-3-azabicyclo[3.1.0]hexan-6-yl]methyl]imidazole-4-carboxamide, (R)-2-(4-(phenyl(3-(trifluoromethyl)phenyl)methyl) piperazin-1-yl) acetic acid and (methylamino) acetic acid.

28. The combination according to claim 27, wherein the GlyT1 inhibitor is N-methyl-N-[(3R)-3-phenyl-3-[4-(trifluoromethyl) phenoxy]propyl]-glycine.

29. The combination according to claim 19, wherein the GlyT inhibitor is a GlyT2 inhibitor.

30. The combination according to claim 29, wherein the GlyT2 inhibitor is selected from the group consisting of N-[1-(dimethylamino)cyclopentyl]methyl]-3,5-dimethoxy-4-(phenylmethoxy)benzamide hydrochloride, 4-butoxy-N-([4-(dimethylamino) oxan-4-yl]methyl)-3,5-dimethoxybenzamide, VVZ-368, and [(5Z,8Z,11Z, 14Z)-Icosa-5,8,11,14-tetraenamido]acetic acid.

31. The combination according to claim 30, wherein the GlyT2 inhibitor is N-[1-(dimethylamino)cyclopentyl]methyl]-3,5-dimethoxy-4-(phenylmethoxy)benzamide hydrochloride.

32. A method for treatment of schizophrenia comprising administering a combination according to claim 19 to a subject suffering from schizophrenia.

33. The method according to claim 32, wherein the combination does not exacerbate positive symptoms of schizophrenia.

34. A method for treatment of negative symptoms of schizophrenia comprising administering a combination according to claim 19 to a subject suffering from schizophrenia.

35. The method according to claim 34, wherein the combination does not exacerbate positive symptoms of schizophrenia.
